(19) United States
(12) Patent Application Publication
(10) Pub. No.: US 2020/0246263 A1
(43) Pub. Date: Aug. 6, 2020

(54) STABLE LIQUID COMPOSITIONS OF PEMETREXED

(71) Applicant: FRESENIUS KABI ONCOLOGY LIMITED, New Delhi (IN)

(72) Inventors: Dhiraj KHATTAR, Gurgaon, Haryana (IN); Rajesh KHANNA, Gurgaon, Haryana (IN); Abhilasha YADAV, Gurgaon, Haryana (IN); Vikas BHANDARI, Gurgaon, Haryana (IN); Sameer Ramanlal MODI, Gurgaon, Haryana (IN); Hemlata, Gurgaon, Haryana (IN)

(73) Assignee: FRESENIUS KABI ONCOLOGY LIMITED, New Delhi (IN)

(21) Appl. No.: 16/641,894

(22) PCT Filed: Aug. 28, 2018
(86) PCT No.: PCT/IB2018/056545

§ 371 Date: Feb. 25, 2020

(30) Foreign Application Priority Data
Aug. 29, 2017 (IN) 201711030501

(51) Int. Cl. A61K 9/08; A61K 47/18; A61K 47/40; A61K 31/519; A61K 9/00

(52) U.S. Cl. CPC A61K 9/08; A61K 47/18; A61K 9/0019; A61K 31/519; A61K 47/40

(57) ABSTRACT

The present invention relates to a stable liquid pharmaceutical composition of pemetrexed for parenteral administration. The invention provides composition comprising pemetrexed diacid, an organic amine and cyclodextrin. The composition may further comprise an inert gas. The composition can be ready to use infusion solution of pemetrexed diacid or liquid concentrate formulation to be diluted before administration to the patient. The present invention further relates to a process for manufacturing the compositions as well as use of the compositions of the invention for the treatment of malignant pleural mesothelioma and non-small cell lung cancer.

### FIELD OF THE INVENTION

The present invention relates to a stable liquid pharmaceutical composition comprising pemetrexed. The invention further relates to a process for manufacturing the composition as well as use of the composition of the invention.

### BACKGROUND OF THE INVENTION

Pemetrexed belongs to the class of chemotherapy drugs called folate antimetabolites and is used in the treatment of malignant pleural mesothelioma and non-small cell lung cancer either as monotherapy or as combination therapy with cisplatin. Pemetrexed has the chemical name N-{4-[2-(2-Amino-4-oxo-4,7-dihydro-1H-pyrrolo[2,3-d]pyrimidin-5-yl)ethyl]benzoyl}-L-glutamic acid and is represented by the chemical formula as formula (I) below, as first disclosed in U.S. Pat. No. 5,344,932.

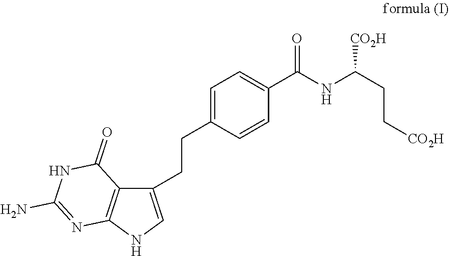 [SMILES: Nc1nc2[nH]cc(CCc3ccc(C(=O)N[C@@H](CCC(=O)O)C(=O)O)cc3)c2c(=O)[nH]1]

By inhibiting thymidylate synthase (TS), dihydrofolate reductase (DHFR), and glycinamide ribonucleotide formyl transferase (GARFT), and hence the formation of precursor purine and pyrimidine nucleotides, pemetrexed prevents the formation of the DNA and RNA required for the growth and survival of both normal cells and cancer cells.

Pemetrexed disodium salt heptahydrate represented by formula (II) is marketed by Eli Lilly and Company under the trade name ALIMTA® as sterile lyophilized powder for intravenous administration.

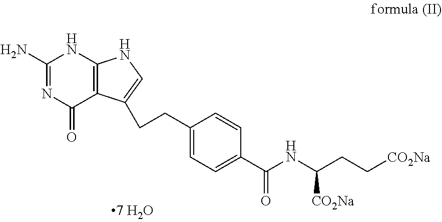

The Eli Lilly commercial product is reported to contain pemetrexed disodium heptahydrate, mannitol, sodium hydroxide, and hydrochloric acid. ALIMTA® is supplied as powder for injection which requires a reconstitution and dilution step before administration to the patient. Chemical and physical stability of reconstituted and infusion solutions of ALIMTA® were demonstrated up to 24 hours following initial reconstitution, when stored at a temperature of 2 to 8° C.

A ready to use infusion solution of pemetrexed diacid or liquid concentrate formulation to be diluted before administration to the patient that could be stored at room temperature for longer period of time is particularly desired for pemetrexed, wherein such ready-to-use compositions provide easier and safer manufacturing and handling during storage and distribution. It is further desirable if the stable pharmaceutical composition can be prepared without the use of freeze drying techniques and eliminating several steps during final administration. The desired liquid formulation can offer enhanced safety for caregiver handling of the cytotoxic materials. Further, a stable, ready-to-use pharmaceutical composition is more acceptable to the patient.

Formulating a liquid composition of pemetrexed or its salts is particularly challenging because of the tendency of pemetrexed and it salts to degrade. This problem is especially exacerbated when pemetrexed or its salts are present in solution, as its degradation or discolouration occurs much more readily due to oxidation or hydrolysis. The degradation on storage of pemetrexed or pemetrexed salts can lead to a significant visible colour change which leads to lesser patient compliance.

Apart from this, another problem observed with the liquid compositions of pemetrexed is precipitation of excipients in the vials during storage. Over time, the increased concentration of degradation products leads to a diminished activity and quality of the product.

Prior art liquid concentrate formulations have suffered from particulate matter in the drug vials which potentially compromises their safety and might necessitate tedious measures like filtration.

This shows, how critical it is to manufacture a stable liquid composition of pemetrexed or its pharmaceutically acceptable salts which is ready to use/dilute. More specifically, it is challenging to produce a room temperature stable liquid product of pemetrexed at large/commercial scale.

Attempts to prepare stable liquid compositions comprising pemetrexed have been made in the prior art.

U.S. Pat. No. 6,686,365 discloses a stable ready-to-use (RTU) formulation of pemetrexed which is developed by using antioxidants/amino acids like L-cysteine, monothioglycerol and thioglycolic acid that can be stored at temperatures above 4° C. in a highly concentrated state.

Previous attempts have been made to stabilize pemetrexed with cyclodextrin but could not provide an ideal approach to stabilizing pemetrexed at room temperature for longer storage time periods.

International patent application WO 2013/179310 A1 claims storage stable concentrated aqueous parenteral composition comprising pemetrexed disodium and at least one stability enhancing adjuvant such as cyclodextrin derivatives and method of preparing these compositions. The formulations claimed in WO 2013/179310 A1 are shown to be stable at refrigerate temperature (2 to 8° C.). The pemetrexed formulations disclosed in this application were found unstable at room temperature when reproduced.

Chinese patent application CN 102846563 discloses a pemetrexed disodium lyophilized powder, comprising pemetrexed disodium, mannitol, [beta]-cyclodextrin which needs to be reconstituted before administration to the patient.

None of these prior art approaches provides a satisfactory solution of providing a simple liquid formulation of pemetrexed or its salts which is room temperature stable and prevents the formation of particulate matter in the finished vials.

There is a need to provide further stable formulations of pemetrexed or pemetrexed salts which are room temperature stable and devoid of any particulate matter during storage. It is therefore an object of the present invention to provide formulations of pemetrexed or its salts, that are stable at room temperature and which can be easily manufactured and readily used.

### SUMMARY OF THE INVENTION

The present invention provides stable liquid composition of pemetrexed, wherein the composition is room temperature stable. Thus, in one aspect of the present invention, there is provided a liquid pharmaceutical composition for parenteral administration comprising:

a) pemetrexed diacid;

b) an organic amine; and

c) cyclodextrin;

In a particular aspect, the composition of the present inventions comprises pemetrexed diacid, tromethamine, cyclodextrin in suitable quantities to stabilize the composition in aqueous solutions. The composition further comprises inert gas selected from argon, helium and nitrogen. Preferably, the inert gas is nitrogen. The compositions of the present invention further comprise optionally other excipients.

In a further aspect, the present invention relates to the composition, wherein said the concentration of pemetrexed diacid is from about 2.5 mg/ml to about 50 mg/ml.

In yet another aspect, the invention relates to the composition, wherein the molar ratio of pemetrexed diacid to cyclodextrin is in range of 1:0.1 to 1:10, preferably 1:0.5 to 1:5, such as 1:2 to 1:5.

In another aspect, the present invention relates to the composition comprising;

a) pemetrexed diacid preferably in an amount of 1-50 mg,

b) tromethamine preferably in an amount of 1-150 mg, and

c) hydroxypropyl-β-cyclodextrin preferably in an amount of 40-500 mg.

The present invention relates to the composition further comprising one or more pharmaceutically acceptable excipients selected from buffer, organic solvent, chelating agent, antioxidant and solubilizer.

In a further aspect, the compositions of the present invention are ready to use or concentrated liquid which can be further diluted with an infusion solution, for example a saline or dextrose solution.

In another aspect, the present invention relates to a process for manufacturing the liquid pharmaceutical composition for parenteral administration comprising the steps of:

a) purging inert gas in the water for injection until the dissolved oxygen content of water is less than 7 mg/L, preferably less than 3 mg/L at 25° C.,

b) dissolving cyclodextrin in water for injection of step a)

c) adding an organic amine to the above solution of step b),

d) adding pemetrexed diacid to the above mixture and dissolving and optionally adjusting the pH of the solution to about 6.0-8.0.

In yet another aspect, the present invention relates to the composition that is substantially free from any particulate matter in the sealed container.

The present invention further relates to the use of the composition of the invention for the treatment of malignant pleural mesothelioma and non-small cell lung cancer.

In a further aspect, the present invention relates to the composition which is room temperature stable.

Particularly preferred embodiments are set forth in the claims.

### DETAILED DESCRIPTION

While this specification concludes with claims particularly pointing out and distinctly claiming that, which is regarded as the invention, it is anticipated that the invention can be more readily understood through reading the following detailed description of the invention and study of the included examples.

The inventors of the present invention found that several stabilization techniques failed to provide sufficient stable liquid compositions of pemetrexed. As the compound has to be administered by infusion techniques, it must be in a readily available form such as ready to dilute in an infusion liquid, or be provided as ready to use infusion solution. The composition must be a clear solution and the active pharmaceutical ingredient must be homogeneously dissolved with other excipients without any precipitation on storage. Another challenge is preparing such aqueous based compositions of pemetrexed which is room temperature stable. In a preferred embodiment the compositions of present invention are substantially free from any particulate matter in the sealed container.

The inventors of the present application have unexpectedly found that pemetrexed formulations containing organic amine in combination with cyclodextrin have shown room temperature stability to the active pharmaceutical compound even in aqueous formulations.

In particular, the combination of organic amine with cyclodextrin might have formed a ternary complex with pemetrexed diacid and this complexation reaction would have provided the extra stability to the formulation due to shielding of reactive sites of pemetrexed diacid. Stability is further enhanced by purging nitrogen to the water for injection to be used for preparation of the infusion solution. The inventors of present invention have thus prepared a stable pharmaceutical composition of pemetrexed diacid which is room temperature stable.

One of the advantages of the compositions of the invention is that they have sufficient long-term stability at room temperature which is defined as less than 2 percent of total impurities after at least about 12 months of storage at a temperature of 25±5° C., which indicates sufficient stability upon storage.

As used herein, the term “pemetrexed” includes pemetrexed diacid or pharmaceutically acceptable salts thereof.

As used herein, the term “pemetrexed diacid” refers to a compound named N-{4-[2-(2-Amino-4-oxo-4,7-dihydro-1H-pyrrolo[2,3-d]pyrimidin-5-yl)ethyl]benzoyl}-L-glutamic acid, represented by the formula (I). Specifically, the term refers to a multitargeted antifolate that exhibits anticancer effects against various cancers, including malignant pleural mesothelioma and non-small cell lung cancer.

As used herein, the term “pharmaceutically acceptable salt” refers to a salt prepared according to a conventional method known in the art includes but not limited to disodium, dipotassium or ditromethamine salt.

The present invention is directed to room temperature stable liquid pharmaceutical composition for parenteral administration comprising pemetrexed diacid, organic amine and cyclodextrin. The composition may further comprise an inert gas. The compositions of the present invention may be ready-to-use or liquid concentrates which are ready-to-dilute with the infusion solutions such as a saline or dextrose solution. The compositions of the present invention are therefore advantageous because they provide enhanced convenience in handling and administration.

The compositions may be presented in a single vial presentation having pemetrexed diacid concentrations in the range of 2.5 to 50 mg/ml, preferably 5-40 mg/ml, more preferably 10-30 mg/ml, of which the preferred concentration is 25 mg/ml.

These pharmaceutical compositions may then be administered via intravenous infusion to treat patients suffering from malignant pleural mesothelioma and for second-line treatment of non small cell lung cancer which is the approved indication of pemetrexed.

The stable ready-to-use pharmaceutical composition of pemetrexed diacid is usually solvated in aqueous solvent comprising water for injection. In one embodiment of the present invention, the ready-to-use pharmaceutical composition of pemetrexed diacid has a pH between about 4 and about 9, preferably between about 5 and about 8 and more preferably in the range of about 6.0 and about 8.0. The pH of such ready-to-use pharmaceutical compositions of pemetrexed diacid may be adjusted with a pharmacologically acceptable pH adjusting agent such as an acid, base, buffer or combination thereof. In an embodiment of the present invention the pH adjusting agent is hydrochloric acid or sodium hydroxide, or a combination thereof. The hydrochloric acid or sodium hydroxide may be in any suitable form, such as a 1 N solution.

In certain preferred embodiments of the present invention, the compositions are ready to use or concentrated liquid ready to be diluted with a suitable infusion solution before administration to the patient, wherein the infusion solution is selected from saline or dextrose solution.

The composition comprises one of the mentioned organic basic amine compounds or a combination thereof. Such compounds include but are not limited to diethanolamine, tris-(hydroxymethyl)aminomethane and meglumine, preferably tromethamine. In preferred embodiments, the total amount of the one or more basic organic amine in the present invention is in the range corresponding to 1 to 150 mg/ml, preferably 10 to 40 mg/ml, more preferably 15 to 35 mg/ml.

In certain preferred embodiments of the present invention, the composition comprises cyclodextrin which includes but is not limited to β-cyclodextrin, hydroxypropyl-β-cyclodextrin, sulfobutylether-β-cyclodextrin, α-cyclodextrin and γ-cyclodextrin, preferably hydroxypropyl-β-cyclodextrin. In preferred embodiments, the cyclodextrin derivative is present in an amount from about 40 mg/ml to about 500 mg/ml, preferably from about 100 mg/ml to about 350 mg/ml. More preferably, the concentration of cyclodextrin derivative in the aqueous parenteral compositions according to the invention is from about 200 mg ml to about 300 mg/ml. The preferred molar ratio of pemetrexed diacid to cyclodextrin according to present invention is in range of 1:0.1 to 1:10, more preferably 1:0.5 to 1:5, such as 1:2 to 1:5.

In another embodiment of the present invention, so as to minimize oxidation of the sensitive material it is also desirable to remove headspace oxygen or moisture or both during the formulation or from the sealable vessel as quickly as possible. This may be aided by, for example, purging the water for injection or sealable container with a gas which is substantially oxygen-free, or substantially moisture free, or substantially oxygen and moisture free before, during or after step, or any combination thereof. Inert gas is purged during manufacturing in the water for injection until the dissolved oxygen content of water is less than 7 mg/L, preferably less than 3 mg/L at 25° C. The headspace of product vials is also blanketed with nitrogen to achieve headspace oxygen content less than 8%, preferably less than 2% to minimize degradation during storage.

The gas used for purging the sealable container may be any appropriate inert gas known to those in the art, such as argon, helium or nitrogen, or a mixture thereof. The most preferred inert gas is nitrogen.

Further one or more pharmaceutically acceptable excipients may as well be included in the compositions of the invention, such as but not limited to buffers, organic solvents, chelating agents, antioxidants, preservatives and solubilizers. Buffer including but not limited to citrate, phosphate, arginate, acetate, glutamate, lactobionate, tartarate, and a mixture thereof. Organic solvent including but not limited to glycerol, poly ethylene glycol (PEG 300, PEG 400), propylene glycol (PG), ethanol, dimethyl acetamide (DMA) and a mixture thereof. Chelating agents including but not limited to ethylenediaminetetraacetic acid (EDTA), sodium citrate. Antioxidant including but not limited to L-cysteine, methionine, monothioglycerol, sodium metasulphite, sodium bi sulphite or a mixture thereof. Solubilizers including but not limited to surfactants/emulsifiers such as polysorbates, polyoxyethylated castor oil, lecithine, polymers such as povidone (PVP), poloxamer, and hydrotropes such as sodium benzoate, sodium salicylate, sodium benzene sulphonate, p-amino benzoic acid hydrochloride, procaine hydrochloride, caffeine, sodium alkanoate, sodium p-toluenesulfonate and sodium xylene sulfonate. Antibacterial preservatives including one or more of phenylmercuric nitrate, thiomersal, benzalkonium chloride, benzethonium chloride, phenol, cresol and chlorobutanol.

The Present Invention Includes Inter Alia the Following Aspects:

In a first aspect, the present invention relates to a liquid pharmaceutical composition for parenteral administration comprising:

a) pemetrexed diacid;

b) an organic amine; and

c) cyclodextrin.

In a second aspect, the present invention relates to the composition according to aspect 1, wherein said the concentration of pemetrexed diacid is from about 2.5 mg/ml to about 50 mg/ml.

In a third aspect, the present invention relates to the composition according to aspects 1 and 2, wherein the composition further comprises an inert gas.

In a fourth aspect, the present invention relates to the composition according to aspect 3, wherein the inert gas is selected from nitrogen, argon and helium, preferably nitrogen.

In a fifth aspect, the present invention relates to the composition according to aspect 1, wherein the molar ratio of pemetrexed diacid to cyclodextrin is in range of 1:0.1 to 1:10, preferably 1:0.5 to 1:5, such as 1:2 to 1:5.

In a sixth aspect, the present invention relates to the composition according to any of the preceding aspects, wherein the concentration of organic amine is from about 1 to 150 mg/ml.

In a seventh aspect, the present invention relates to the composition according to any of the preceding aspects, wherein the organic amine is preferably tromethamine.

In an eight aspect, the present invention relates to the composition according to any of the preceding aspects, wherein the cyclodextrin is selected from β-cyclodextrin, hydroxypropyl-β-cyclodextrin, sulfobutylether-β-cyclodextrin, α-cyclodextrin and γ-cyclodextrin preferably hydroxypropyl-β-cyclodextrin.

In a ninth aspect, the present invention relates to the composition according to any of the preceding aspects, wherein the concentration of cyclodextrin is from about of 40-500 mg/ml.

In a tenth aspect, the present invention relates to the composition according to any of the preceding aspects, further comprising one or more pharmaceutically acceptable excipients selected from buffer, organic solvent, chelating agent, antioxidant and solubilizer.

In an eleventh aspect, the present invention relates to the composition according to aspect 10, wherein the buffer is selected from the group consisting of citrate, phosphate, acetate, glutamate, lactobionate, and a mixture thereof.

In one embodiment, the present invention relates to the composition wherein the buffer is citrate buffer.

In another embodiment, the present invention relates to the composition wherein the buffer is phosphate buffer.

In yet another embodiment, the present invention relates to the composition wherein the buffer is acetate buffer.

In another embodiment, the present invention relates to the composition wherein the buffer is glutamate buffer.

In yet another embodiment, the present invention relates to the composition wherein the buffer is glutamate buffer.

In a twelfth aspect, the present invention relates to the composition according to aspect 10, wherein the organic solvent is selected from the group consisting of glycerol, poly ethylene glycol (PEG 300, PEG 400), propylene glycol (PG), ethanol, dimethyl acetamide (DMA) and a mixture thereof.

In one embodiment, the present invention relates to the composition wherein the organic solvent is glycerol.

In another embodiment, the present invention relates to the composition wherein the organic solvent is poly ethylene glycol (PEG 300, PEG 400).

In yet another embodiment, the present invention relates to the composition wherein the organic solvent is propylene glycol (PG).

In another embodiment, the present invention relates to the composition wherein the organic solvent is ethanol.

In yet another embodiment, the present invention relates to the composition wherein the organic solvent is dimethyl acetamide (DMA).

In a thirteenth aspect, the present invention relates to the composition according to aspect 10, wherein the chelating agent is selected from the group consisting of ethylenediaminetetraacetic acid (EDTA), sodium citrate and a mixture thereof.

In one embodiment, the present invention relates to the composition wherein the chelating agent is ethylenediaminetetraacetic acid (EDTA).

In another embodiment, the present invention relates to the composition wherein the chelating agent is sodium citrate

In a fourteenth aspect, the present invention relates to the composition according to aspect 10, wherein the antioxidant is selected from the group consisting of methionine, sodium metasulphite, sodium bisulphite and a mixture thereof.

In one embodiment, the present invention relates to the composition wherein the antioxidant is methionine.

In another embodiment, the present invention relates to the composition wherein the antioxidant is sodium metasulphite.

In another embodiment, the present invention relates to the composition wherein the antioxidant is sodium bisulphite.

In a fifteenth aspect, the present invention relates to the composition according to aspect 10, wherein the solubilizer is selected from the group consisting of povidone (PVP), lecithine, sodium benzoate, poloxamer and a mixture thereof.

In a sixteenth aspect, the present invention relates to the composition according to any of the preceding aspects for treating a subject suffering from malignant pleural mesothelioma and refractory non small cell lung cancer.

In a seventeenth aspect, the present invention relates to the composition according to any of the preceding aspects, wherein the composition is ready to use or ready to be diluted with a suitable infusion solution before administration to the patient.

In an eighteenth aspect, the present invention relates to the composition according to aspect 17, wherein the infusion solution is selected from saline or dextrose solution.

In a nineteenth aspect, the present invention relates to the composition according to aspect 1, is substantially free from any particulate matter in the sealed container.

In a twentieth aspect, the present invention relates to the composition according to any of the preceding aspects comprising;

a) pemetrexed diacid preferably in an amount of 1-50 mg,

b) tromethamine preferably in an amount of 1-150 mg,

c) hydroxypropyl-β-cyclodextrin preferably in an amount of 40-500 mg.

In a twenty one aspect, the present invention relates to a process for manufacturing the liquid pharmaceutical composition for parenteral administration according to any of the preceding aspects comprising the steps of:

a) purging inert gas in the water for injection until the dissolved oxygen content of water is less than 7 mg/L, preferably less than 3 mg/L at 25° C.,

b) dissolving cyclodextrin in water for injection of step a)

c) adding an organic amine to the above solution of step b),

d) adding pemetrexed diacid to the above mixture and dissolving and optionally adjusting the pH of the solution to about 6.0-8.0,

In a twenty two aspect, the present invention relates to a process according to aspect 21, wherein the inert gas is nitrogen.

In a twenty three aspect, the present invention relates to a process according to aspect 21, wherein the cyclodextrin is hydroxypropyl-β-cyclodextrin.

In a twenty four aspect, the present invention relates to a process according to aspect 21, wherein the organic amine is tromethamine.

In a twenty five aspect, the present invention relates to a process for manufacturing the liquid pharmaceutical composition for parenteral administration according to aspect 21 comprising the additional following subsequent to steps a)-d):

e) filtering the solution and filling in vials,

f) headspace blanketing with nitrogen and

g) stoppering and sealing the vials.

In a twenty six aspect, the present invention relates to the composition according to aspects 1 to 20, wherein the composition is room temperature stable.

In a twenty seven aspect, the present invention relates to the composition further comprising a preservative selected from the group consisting of phenylmercuric nitrate, thiomersal, benzalkonium chloride, benzethonium chloride, phenol, cresol and chlorobutanol or a mixture thereof.

In another aspect, the formulation can be sterilized by filteration, aseptic processing, termination sterilization, by combination of such techniques, or any other suitable sterilization process known in the art.

### EXAMPLES

The invention is further illustrated by way of the following examples, which in no way should be construed as limiting the scope of the invention. The process of manufacturing of the compositions below was carried at room temperature (25° C.)

### Examples 1, 2, 3 and 4

TABLE 1
Composition of pemetrexed injection

| | Quantity per mL | | | |
|---|---|---|---|---|
| Ingredient | Example 1 | Example 2 | Example 3 | Example 4 |
| Pemetrexed | 25 mg | 25 mg | 25 mg | 25 mg |
| diacid | | | | |
| Tromethamine | 15 mg | 15 mg | 15 mg | 15 mg |
| HPβCD | 84 mg | 222 mg | 420 mg | 241 mg |
| Tromethamine | q.s. | q.s. | q.s. | q.s. |
| Hydrochloric | q.s. | q.s. | q.s. | q.s. |
| acid | | | | |
| Water | q.s. to 1 mL | q.s. to 1 mL | q.s. to 1 mL | q.s. to 1 mL |
| Nitrogen | q.s. | q.s. | q.s. | q.s. |

Manufacturing Process (for Example 1, 2, 3 and 4)

A suitable quantity of water in a manufacturing vessel was taken. Nitrogen was purged into water until the dissolved oxygen content of the water became less than 7 mg/L, preferably less than 3 mg/L. After nitrogen bubbling, the required quantity of HPβCD was added and dissolved. Once a clear solution was obtained, tromethamine was added and dissolved. After addition of tromethamine, pemetrexed diacid was added and allowed to stir until a clear solution was obtained. If required, the pH of solution was adjusted to 6.0-8.0 with the help of 10% w/v tromethamine solution or 1% v/v hydrochloric acid solution. The volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed.

### Example 5

TABLE 2
Composition of pemetrexed injection

| Ingredient | Quantity per mL |
|---|---|
| Pemetrexed diacid | 25 mg |
| Tromethamine | 15 mg |
| HPβCD | 222 mg |
| Methionine | 1 mg |
| Tromethamine | q.s. |
| Hydrochloric acid | q.s. |
| Water | q.s. to 1 mL |
| Nitrogen | q.s. |

Manufacturing Process

A suitable quantity of water in a manufacturing vessel was taken. Nitrogen was purged into the water until the dissolved oxygen content of the water became less than 7 mg/L, preferably less than 3 mg/L. After nitrogen bubbling, the required quantity of HPβCD followed by methionine were added and dissolved. Once a clear solution was obtained, tromethamine was added and dissolved. After addition of tromethamine, pemetrexed diacid was added and allowed to stir until a clear solution was obtained. If required, the pH of solution was adjusted to 6.0-8.0 with the help of 10% w/v tromethamine solution or 1% v/v hydrochloric acid solution. The volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed.

### Example 6

TABLE 3
Composition of pemetrexed injection

| Ingredient | Quantity per mL |
|---|---|
| Pemetrexed diacid | 25 mg |
| Tromethamine | 15 mg |
| HPβCD | 222 mg |
| Glycerol | 90 mg |
| Tromethamine | q.s. |
| Hydrochloric acid | q.s. |
| Water | q.s. to 1 mL |
| Nitrogen | q.s. |

Manufacturing Process

A suitable quantity of water in a manufacturing vessel was taken. Nitrogen was purged into water until dissolved oxygen content of water became less than 7 mg/L, preferably less than 3 mg/L. After nitrogen bubbling, required quantity of HPβCD was added and dissolved. Once a clear solution was obtained, tromethamine was added and dissolved. After addition of tromethamine, pemetrexed diacid was added and allowed to stir until clear solution was obtained. Required quantity of glycerol was added and stirred vigorously for proper mixing of solvent system. If required, the pH of solution was adjusted to 6.0-8.0 with the help of 10% w/v tromethamine solution or 1% v/v hydrochloric acid solution. Volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed.

### Example 7

TABLE 4
Composition of pemetrexed injection

| Ingredient | Quantity per mL |
|---|---|
| Pemetrexed diacid | 25 mg |
| Tromethamine | 35 mg |
| Citric acid | 10 mg |
| HPβCD | 222 mg |
| Tromethamine | q.s. |
| Hydrochloric acid | q.s. |
| Water | q.s. to 1 mL |
| Nitrogen | q.s. |

Manufacturing Process

A suitable quantity of water in a manufacturing vessel was taken. Nitrogen was purged into the water until the dissolved oxygen content of the water became less than 7 mg/L, preferably less than 3 mg/L. After nitrogen bubbling, the required quantity of HPβCD and citric acid was added and dissolved. Once a clear solution was obtained, tromethamine was added and dissolved. After dissolution of tromethamine, pemetrexed diacid was added and allowed to stir until a clear solution was obtained. If required, the pH of solution was adjusted to 6.0-8.0 with the help of 10% w/v tromethamine solution or 1% v/v hydrochloric acid solution. The volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed.

### Example 8

TABLE 5
Composition of pemetrexed injection

| Ingredient | Quantity per mL |
|---|---|
| Pemetrexed diacid | 25 mg |
| Tromethamine | 15 mg |
| HPβCD | 222 mg |
| Arginine | 10 mg |
| Tromethamine | q.s. |
| Hydrochloric acid | q.s. |
| Water | q.s. to 1 mL |
| Nitrogen | q.s. |

Manufacturing Process

A suitable quantity of water in a manufacturing vessel was taken. Nitrogen was purged into water until the dissolved oxygen content of the water became less than 7 mg/L, preferably less than 3 mg/L. After nitrogen bubbling, required quantity of HPβCD was added and dissolved. Once a clear solution was obtained, tromethamine and arginine was added and dissolved. Pemetrexed diacid was added and allowed to stir until a clear solution was obtained. If required, the pH of solution was adjusted to 6.0-8.0 with the help of 10% w/v tromethamine solution or 1% v/v hydrochloric acid solution. The volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed.

### Example 9

TABLE 6
Composition of pemetrexed injection

| Ingredient | Quantity per mL |
|---|---|
| Pemetrexed diacid | 25 mg |
| Tromethamine | 15 mg |
| HPβCD | 222 mg |
| Potassium dihydrogen phosphate | 10 mg |
| Tromethamine | q.s. |
| Hydrochloric acid | q.s. |
| Water | q.s. to 1 mL |
| Nitrogen | q.s. |

Manufacturing Process

Suitable quantity of water in a manufacturing vessel was taken. Nitrogen was purged into water until dissolved oxygen content of water became less than 7 mg/L, preferably less than 3 mg/L. After nitrogen bubbling, required quantity of HPβCD was added and dissolved. Once a clear solution was obtained, tromethamine and potassium dihydrogen phosphate was added and dissolved. pemetrexed diacid was added and allowed to stir until clear solution was obtained. If required, the pH of solution was adjusted to 6.0-8.0 with the help of 10% w/v tromethamine solution or 1% v/v hydrochloric acid solution. Volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed.

### Example 10

TABLE 7
Composition of pemetrexed injection

| Ingredient | Quantity per mL |
|---|---|
| Pemetrexed | 25 mg |
| diacid | |
| Tromethamine | 15 mg |
| HPβCD | 222 mg |
| Methionine | 1 mg |
| Glycerol | 90 mg |
| Tromethamine | q.s. |
| Hydrochloric | q.s. |
| acid | |
| Water | q.s. to 1 mL |
| Nitrogen | q.s. |

Manufacturing Process

Suitable quantity of water in a manufacturing vessel was taken. Nitrogen was purged into water until dissolved oxygen content of water became less than 7 mg/L, preferably less than 3 mg/L. After nitrogen bubbling, required quantity of HPβCD was added and dissolved. Once a clear solution was obtained, tromethamine and methionine was added and dissolved. pemetrexed diacid was added and allowed to stir until clear solution was obtained followed by addition and mixing of glycerol. If required, the pH of solution was adjusted to 6.0-8.0 with the help of 10% w/v tromethamine solution or 1% v/v hydrochloric acid solution. Volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed.

### Example 11

TABLE 8
Composition of pemetrexed injection

| Ingredient | Quantity per mL |
|---|---|
| Pemetrexed diacid | 25 mg |
| Tromethamine | 15 mg |
| HPβCD | 222 mg |
| Methionine | 1 mg |
| Glycerol | 90 mg |
| Tromethamine | q.s. |
| Hydrochloric acid | q.s. |
| Water | q.s. to 1 mL |
| Nitrogen | q.s. |

Terminal sterilization at 121° C. for 15
min

Manufacturing Process

Suitable quantity of water in a manufacturing vessel was taken. Nitrogen was purged into water until dissolved oxygen content of water became less than 7 mg/L, preferably less than 3 mg/L. After nitrogen bubbling, required quantity of HPβCD was added and dissolved. Once a clear solution was obtained, tromethamine and methionine was added and dissolved. Pemetrexed diacid was added and allowed to stir until clear solution was obtained followed by addition and mixing of glycerol. If required, the pH of solution was adjusted to 6.0-8.0 with the help of 10% w/v tromethamine solution or 1% v/v hydrochloric acid solution. Volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed. The sealed vials were terminally sterilized at 121° C. for 15 min.

### Example 12

TABLE 9
Composition of pemetrexed injection

| Ingredient | Quantity per mL |
|---|---|
| Pemetrexed | 25 mg |
| diacid | |
| Tromethamine | 15 mg |
| Methionine | 1 mg |
| Tromethamine | q.s. |
| Hydrochloric acid | q.s. |
| Water | q.s. to 1 mL |
| Nitrogen | q.s. |

Manufacturing Process

Suitable quantity of water in a manufacturing vessel was taken. Nitrogen was purged into water until dissolved oxygen content of water became less than 7 mg/L, preferably less than 3 mg/L. After nitrogen bubbling, required quantity of tromethamine and methionine was added and dissolved. Pemetrexed diacid was added and allowed to stir until clear solution was obtained. If required, the pH of solution was adjusted to 6.0-8.0 with the help of 10% w/v tromethamine solution or 1% v/v hydrochloric acid solution. Volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed.

### Example 13

TABLE 10
Composition of pemetrexed injection

| Ingredient | Quantity per mL |
|---|---|
| Pemetrexed diacid | 25 mg |
| Tromethamine | 15 mg |
| Glycerol | 90 mg |
| Tromethamine | q.s. |
| Hydrochloric acid | q.s. |
| Water | q.s. to 1 mL |
| Nitrogen | q.s. |

Manufacturing Process

Suitable quantity of water in a manufacturing vessel was taken. Nitrogen was purged into water until dissolved oxygen content of water became less than 7 mg/L, preferably less than 3 mg/L. After nitrogen bubbling, required quantity of tromethamine was added and dissolved. pemetrexed diacid was added and allowed to stir until clear solution was obtained followed by addition and mixing of glycerol. If required, the pH of solution was adjusted to 6.0-8.0 with the help of 10% w/v tromethamine solution or 1% v/v hydrochloric acid solution. Volume was made up to 100% with water. The drug solution was filtered through a suitable 0.2μ PVDF filter. The filtered solution was filled into vials. The vial headspace was blanketed with nitrogen to achieve headspace oxygen content less than 8%, preferably less than 2%. The vials were stoppered and finally sealed. The sealed vials were terminally sterilized at 121° C. for 15 min.

Characterization Data:

Stability profiles of the pharmaceutical compositions as mentioned in above Examples 1 to 13 have been summarized below in Table 11 to 23, respectively.

TABLE 11
Stability profile of the pharmaceutical composition
as mentioned in Example 1

| | | | | Total |
|---|---|---|---|---|
| Storage | Time | | | impurity |
| condition | point | Description | pH | (%) |
| Initial | | Clear colorless solution, | 6.9 | 0.34 |
| | | free from visible particles | | |
| 40° C./75% | 1 month | Clear colorless solution, | 6.9 | 0.44 |
| RH | | free from visible particles | | |
| | 3 month | Clear light green colored | 7.0 | 0.72 |
| | | solution, free from visible | | |
| | | particles | | |
| 25° C./60% | 1 month | Clear colorless solution, | 6.9 | 0.34 |
| RH | | free from visible particles | | |
| | 3 month | Clear colorless solution, | 7.0 | 0.41 |
| | | free from visible particles | | |

TABLE 12
Stability profile of the pharmaceutical composition as mentioned
in Example 2

| | | | | Total |
|---|---|---|---|---|
| Storage | Time | | | impurity |
| condition | point | Description | pH | (%) |
| Initial | | Clear colorless solution, | 6.9 | 0.31 |
| | | free from visible particles | | |
| 40° C./75% | 1 month | Clear colorless solution, | 6.8 | 0.53 |
| RH | | free from visible particles | | |
| | 3 month | Clear light green colored | 6.9 | 0.69 |
| | | solution, free from visible | | |
| | | particles | | |
| | 6 month | Light green color | 6.9 | 1.20 |
| | | solution, free from visible | | |
| | | particles | | |
| 25° C./60% | 1 month | Clear colorless solution, | 6.9 | 0.40 |
| RH | 1 month | free from visible particles | | |
| | 3 month | Clear colorless solution, | 6.9 | 0.43 |
| | | free from visible particles | | |
| | 6 month | Clear colorless solution' | 6.9 | 0.52 |
| | | free from visible particles | | |
| | 12 month | Clear light green color | 6.9 | 0.73 |
| | | solution free from visible | | |
| | | particles | | |

TABLE 13
Stability profile of the pharmaceutical composition as mentioned
in Example 3

| | | | | Total |
|---|---|---|---|---|
| Storage | Time | | | impurity |
| condition | point | Description | pH | (%) |
| Initial | | Clear colorless solution, | 7.4 | 0.36 |
| | | free from visible particles | | |
| 40° C./75% | 1 month | Clear colorless solution, | 7.4 | 0.48 |
| RH | | free from visible particles | | |
| | 3 month | Clear colorless solution, | 7.7 | 0.78 |
| | | free from visible particles | | |
| 25° C./60% | 1 month | Clear colorless solution, | 7.3 | 0.34 |
| RH | | free from visible particles | | |
| | 3 month | Clear colorless solution, | 7.6 | 0.42 |
| | | free from visible particle | | |

TABLE 14
Stability profile of the pharmaceutical composition as mentioned
in Example 4

| | | | | Total |
|---|---|---|---|---|
| Storage | Time | | | impurity |
| condition | point | Description | pH | (%) |
| Initial | | Clear colorless solution, | 7.3 | 0.36 |
| | | free from visible particles | | |
| 40° C./75% | 1 month | Clear colorless solution, | 7.2 | 0.57 |
| RH | | free from visible particles | | |
| | 3 month | Clear light yellow colored | 7.2 | 0.86 |
| | | solution, free from visible | | |
| | | particles | | |
| 25° C./60% | 1 month | Clear colorless solution, | 7.2 | 0.44 |
| RH | | free from visible particles | | |
| | 3 month | Clear colorless solution, | 7.2 | 0.46 |
| | | free from visible particles | | |

TABLE 15
Stability profile of the pharmaceutical composition as mentioned
in Example 5

| | | | | Total |
|---|---|---|---|---|
| Storage | Time | | | impurity |
| condition | point | Description | pH | (%) |
| Initial | | Clear colorless solution, | 6.9 | 0.33 |
| | | free from visible particles | | |
| 40° C./75% | 1 month | Clear light green colored | 7.0 | 0.44 |
| RH | | solution, free from visible | | |
| | | particles | | |
| | 3 month | Clear light green colored | 6.9 | 0.70 |
| | | solution, free from visible | | |
| | | particles | | |
| 25° C./60% | 1 month | Clear colorless solution, | 7.0 | 0.39 |
| RH | | free from visible particle | | |
| | 3 month | Clear colorless solution, | 6.9 | 0.41 |
| | | free from visible particle | | |

TABLE 16
Stability profile of the pharmaceutical composition as mentioned
in Example 6

| | | | | Total |
|---|---|---|---|---|
| Storage | Time | | | impurity |
| condition | point | Description | pH | (%) |
| Initial | | Clear colorless solution, | 6.8 | 0.37 |
| | | free from visible particles | | |
| 40° C./75% | 1 month | Clear colorless solution, | 6.8 | 0.72 |
| RH | | free from visible particles | | |
| | 3 month | Clear light green colored | 6.9 | 0.84 |
| | | solution, free from visible | | |
| | | particles | | |
| | 6 months | Clear green colored | 6.8 | 1.54 |
| | | solution, free from visible | | |
| | | particles | | |
| 25° C./60% | 1 month | Clear colorless solution, | 6.9 | 0.48 |
| RH | | free from visible particle | | |
| | 3 month | Clear colorless solution, | 6.9 | 0.44 |
| | | free from visible particle | | |
| | 6 months | Clear light green colored | 6.9 | 0.60 |
| | | solution, free from visible | | |
| | | particles | | |

TABLE 17
Stability profile of the pharmaceutical composition as mentioned
in Example 7

| | | | | Total |
|---|---|---|---|---|
| Storage | Time | | | impurity |
| condition | point | Description | pH | (%) |
| Initial | | Clear colorless solution, | 7.1 | 0.37 |
| | | free from visible particles | | |
| 40° C./75% | 1 month | Clear colorless solution, | 7.4 | 0.68 |
| RH | | free from visible particles | | |
| | 3 month | Clear light green colored | 7.1 | 1.23 |
| | | solution, free from visible | | |
| | | particles | | |
| 25° C./60% | 1 month | Clear colorless solution, | 7.2 | 0.46 |
| RH | | free from visible particles | | |
| | 3 month | Clear colorless solution, | 7.1 | 0.62 |
| | | free from visible particle | | |

TABLE 18
Stability profile of the pharmaceutical composition as mentioned
in Example 8

| | | | | Total |
|---|---|---|---|---|
| Storage | Time | | | impurity |
| condition | point | Description | pH | (%) |
| Initial | | Clear colorless solution, | 7.4 | 0.34 |
| | | free from visible particles | | |
| 40° C./75% | 1 month | Clear colorless solution, | 7.4 | 0.51 |
| RH | | free from visible particles | | |
| | 3 month | Clear light green colored | 7.3 | 0.82 |
| | | solution, free from visible | | |
| | | particles | | |
| 25° C./60% | 1 month | Clear colorless solution, | 7.4 | 0.38 |
| RH | | free from visible particles | | |
| | 3 month | Clear colorless solution, | 7.3 | 0.45 |
| | | free from visible particle | | |

TABLE 19
Stability profile of the pharmaceutical composition as mentioned
in Example 9

| | | | | Total |
|---|---|---|---|---|
| Storage | Time | | | impurity |
| condition | point | Description | pH | (%) |
| Initial | | Clear colorless solution, | 7.4 | 0.34 |
| | | free from visible particles | | |
| 40° C./75% | 1 month | Clear colorless solution, | 7.4 | 0.61 |
| RH | | free from visible particles | | |
| | 3 month | Clear light green colored | 7.4 | 1.18 |
| | | solution, free from visible | | |
| | | particles | | |
| 25° C./60% | 1 month | Clear colorless solution, | 7.4 | 0.40 |
| RH | | free from visible particles | | |
| | 3 month | Clear colorless solution, | 7.3 | 0.50 |
| | | free from visible particle | | |

TABLE 20
Stability profile of the pharmaceutical composition as mentioned
in Example 10

| | | | | Total |
|---|---|---|---|---|
| Storage | Time | | | impurity |
| condition | point | Description | pH | (%) |
| Initial | | Clear colorless solution, | 6.9 | 0.35 |
| | | free from visible particles | | |
| 40° C./75% | 1 month | Clear colorless solution, | 7.0 | 0.51 |
| RH | | free from visible particles | | |
| | 3 month | Clear light green colored | 7.0 | 0.90 |
| | | solution, free from visible | | |
| | | particles | | |
| | 6 month | Light green colored | 6.9 | 1.31 |
| | | solution, free from visible | | |
| | | particles | | |
| 25° C./60% | 1 month | Clear colorless solution, | 6.9 | 0.34 |
| RH | | free from visible particles | | |
| | 3 month | Clear colorless solution, | 6.9 | 0.48 |
| | | free from visible particle | | |
| | 6 month | Clear colorless solution, | 7.2 | 0.60 |
| | | free from visible particle | | |

TABLE 21
Stability profile of the pharmaceutical composition as mentioned
in Example 11

| | | | Total |
|---|---|---|---|
| Storage | Time | | impurity |
| condition | point | Description | (%) |
| Initial | | Clear colorless solution, free | 0.66 |
| | | from visible particles | |
| 25° C./60% RH | 1 month | Clear colorless solution, free | 0.64 |
| | | from visible particles | |
| | 3 month | Clear colorless solution, free | 0.75 |
| | | from visible particle | |

TABLE 22
Stability profile of the pharmaceutical composition as mentioned
in Example 12

| | | | | Total |
|---|---|---|---|---|
| Storage | Time | | | impurity |
| condition | point | Description | pH | (%) |
| Initial | | Clear colorless solution, | 7.5 | 0.55 |
| | | free from visible particles | | |
| 40° C./75% | 1 month | Clear light green colored | 7.3 | 1.86 |
| RH | | solution, free from visible | | |
| | | particles | | |
| | 3 month | Clear light green colored | 7.4 | 6.2 |
| | | solution, free from visible | | |
| | | particles | | |
| 25° C./60% | 1 month | Clear light green colored | 7.3 | 0.85 |
| RH | | solution, free from visible | | |
| | | particles | | |
| | 3 month | Clear light green colored | 7.5 | 1.94 |
| | | solution, free from visible | | |
| | | particles | | |
| | 6 month | Clear light green colored | 7.5 | 4.96 |
| | | solution, free from visible | | |
| | | particles | | |

TABLE 23
Stability profile of the pharmaceutical composition as
mentioned in Example 13

| | | | | Total |
|---|---|---|---|---|
| Storage | Time | | | impurity |
| condition | point | Description | pH | (%) |
| Initial | | Clear colorless solution, | 6.9 | 0.28 |
| | | free from visible particles | | |
| 40° C./75% | 1 month | Clear light yellow colored | 6.9 | 0.28 |
| RH | | solution, free from visible | | |
| | | particles | | |
| | 3 month | Clear light green colored | 7.1 | 0.98 |
| | | solution, free from visible | | |
| | | particles | | |
| | 6 month | Clear green colored | 6.9 | 2.20 |
| | | solution, free from visible | | |
| | | particles | | |
| 25° C./60% | 1 month | Clear light yellow colored | 7.0 | 0.21 |
| RH | | solution, free from visible | | |
| | | particles | | |
| | 3 month | Clear light yellow colored | 7.1 | 0.94 |
| | | solution, free from visible | | |
| | | particles | | |
| | 6 month | Clear light green colored | 6.9 | 1.57 |
| | | solution, free from visible | | |
| | | particles | | |
| | 12 month | Clear light green colored | 6.9 | 4.98 |
| | | solution, free from visible | | |
| | | particles | | |

It is apparent from the above characterization data that injectable compositions comprising pemetrexed diacid, cyclodextrin, tromethamine are showing better room temperature stability results in comparison to formulations without these essential features.

Further to evaluate the room temperature stability of prior art formulation of pemetrexed disodium with cyclodextrins, the inventors have reproduced the prior art formulations, but found them to be less stable at room temperature in comparison to the compositions of present invention as shown below:

### Examples 14 and 15

TABLE 24
Prior art compositions of pemetrexed injection with HPβCD

| | Example 14 | | Example 15 | |
|---|---|---|---|---|
| | Quantity | Quantity | Quantity | Quantity |
| Ingredient | per mL | % w/w | per mL | % w/w |
| Pemetrexed | 25 mg | 1 | 25 mg | 1 |
| disodium | | | | |
| HPβCD | 25 mg | 1 | 75 mg | 3 |
| Sodium | q.s | q.s. | q.s. | q.s. |
| hydroxide | | | | |
| Hydrochloric | q.s. | q.s. | q.s | q.s. |
| acid | | | | |
| Water for injection | q.s. to 1 mL | q.s. | q.s. to 1 mL | q.s. |

Manufacturing Process (for Examples 14 and 15)

Suitable quantity of water in a manufacturing vessel was taken. Required quantity of HPβCD was added and dissolved. Once a clear solution was obtained, pemetrexed disodium was added and allowed to stir until clear solution was obtained. If required, the pH of solution was adjusted to 7.0±0.2 with the help of 1% w/v sodium hydroxide solution or 1% v/v hydrochloric acid solution. Volume was made up to 100% with water. The drug solution was filtered through a suitable 0.2μ PVDF filter. The filtered solution was filled into vials. The vials were stoppered and finally sealed.

TABLE 25
Stability profile of the pharmaceutical composition as mentioned
in Example 14

| | | | | Total |
|---|---|---|---|---|
| Storage | Time | | | impurity |
| condition | point | Description | pH | (%) |
| Initial | | Clear colorless solution, free | 7.2 | 0.58 |
| | | from visible particles | | |
| 40° C./75% | 1 month | Clear light green colored | 6.8 | 5.21 |
| RH | | solution, free from visible | | |
| | | particles | | |
| 25° C./60% | 1 month | Clear light green colored | 6.9 | 1.50 |
| RH | | solution, free from visible | | |
| | | particles | | |

TABLE 26
Stability profile of the pharmaceutical composition as
mentioned in Example 15

| | | | | Total |
|---|---|---|---|---|
| Storage | Time | | | impurity |
| condition | point | Description | pH | (%) |
| Initial | | Clear colorless solution, free | 7.1 | 0.34 |
| | | from visible particles | | |
| 40° C./75% | 1 month | Clear light green colored | 7.1 | 1.79 |
| RH | | solution, free from visible | | |
| | | particles | | |
| 25° C./60% | 1 month | Clear light green colored | 6.9 | 0.68 |
| RH | | solution, free from visible | | |
| | | particles | | |

1. A liquid pharmaceutical composition for parenteral administration comprising:
a) pemetrexed diacid;
b) an organic amine; and
c) cyclodextrin.

2. The composition according to claim 1, wherein the concentration of pemetrexed diacid is from about 2.5 mg/ml to about 50 mg/ml.

3. The composition according to claim 1, wherein the composition further comprises an inert gas which is nitrogen, argon, or helium.

4. (canceled)

5. The composition according to claim 1, wherein the molar ratio of pemetrexed diacid to cyclodextrin is in the range of 1:0.5 to 1:5.

6. The composition according to claim 1, wherein the concentration of organic amine is from about 1 to 150 mg/ml.

7. The composition according to claim 1, wherein the organic amine is tromethamine.

8. The composition according to claim 1, wherein the cyclodextrin is selected from β-cyclodextrin, hydroxypropyl-β-cyclodextrin, sulfobutylether-β-cyclodextrin, α-cyclodextrin and γ-cyclodextrin.

9. The composition according to claim 1, wherein the concentration of cyclodextrin is from about 40 to 500 mg/ml.

10. The composition according to claim 1, further comprising one or more pharmaceutically acceptable excipients selected from buffer, organic solvent, chelating agent, antioxidant and solubilizer.

11. The composition according to claim 10, wherein the buffer is selected from the group consisting of citrate, phosphate, arginate, acetate, glutamate, lactobionate, and a mixture thereof.

12. The composition according to claim 10, wherein the organic solvent is selected from the group consisting of glycerol, poly ethylene glycol (PEG 300, PEG 400), propylene glycol (PG), ethanol, dimethyl acetamide (DMA) and a mixture thereof.

13. The composition according to claim 10, wherein the chelating agent is selected from the group consisting of ethylenediaminetetraacetic acid (EDTA), sodium citrate and a mixture thereof.

14. The composition according to claim 10, wherein the antioxidant is selected from the group consisting of methionine, sodium metasulphite, sodium bisulphite and a mixture thereof.

15. The composition according to claim 10, wherein the solubilizer is selected from the group consisting of povidone (PVP), lecithine, sodium benzoate, poloxamer and a mixture thereof.

16-18. (canceled)

19. The composition according to claim 1, wherein the composition is substantially free from any particulate matter in the sealed container.

20. The composition according to any of the preceding claims claim 1, comprising;
a) pemetrexed diacid in an amount of 1-50 mg/ml,
b) tromethamine in an amount of 15 to 35 mg/ml, and
c) hydroxypropyl-β-cyclodextrin in an amount of about 200 mg/ml to about 300 mg/ml.

21. A process for manufacturing the liquid pharmaceutical composition for parenteral administration according to claim 1, comprising the steps of:
a) purging inert gas in water for injection until the dissolved oxygen content of water is less than 7 mg/L at room temperature,
b) dissolving cyclodextrin in the water for injection of step a)
c) adding an organic amine to the solution of step b),
d) adding and dissolving pemetrexed diacid to the mixture of step c) at room temperature and, optionally, adjusting the pH of the solution to about 6.0-8.0, thereby manufacturing the liquid pharmaceutical composition for parenteral administration.

22. (canceled)

23. The process according to claim 21, wherein the cyclodextrin is hydroxypropyl-β-cyclodextrin.

24. The process according to claim 21, wherein the organic amine is tromethamine.

25. The process for manufacturing the liquid pharmaceutical composition for parenteral administration according to claim 21 further comprising:
e) filtering the liquid pharmaceutical composition of step d) and filling the filtered liquid pharmaceutical composition into vials,
f) blanketing the headspace of the filled vials with nitrogen and
g) stoppering and sealing the blanketed vials.

26. The composition according to claim 1, wherein the composition is room temperature stable.

27. The composition according to claim 26, wherein the composition comprises not more than 0.75% of total impurities following storage of the composition at 25° C. and 60% relative humidity for 3 months.

28. The composition according to claim 26, wherein the composition comprises less than 2 percent of total impurities following storage of the composition at 25±5° C. for at least 12 months.
